(19) United States
(12) Patent Application Publication
(10) Pub. No.: US 2020/0246494 A1
(43) Pub. Date: Aug. 6, 2020

(54) LU-CONTAINING COMPOSITIONS AND METHODS OF PRODUCING THE SAME

(71) Applicants: L'Air Liquide, Société Anonyme pour I'Etude et I'Exploitation des Procédés Georges Claude, Paris (FR); American Air Liquide, Inc., Fremont, CA (US)

(72) Inventors: Quentin DEMARLY, Lery (FR); Benjamin J. JURCIK, Landenberg, PA (US); Nicolas BLASCO, Grenoble (FR)

(21) Appl. No.: 16/637,299

(22) PCT Filed: Jun. 29, 2018
(86) PCT No.: PCT/US2018/040233

§ 371 Date: Feb. 7, 2020

Related U.S. Application Data
(60) Provisional application No. 62/542,078, filed on Aug. 7, 2017.

(51) Int. Cl. A61K 51/12; A61K 51/04

(52) U.S. Cl. CPC A61K 51/1255; A61K 51/0478

(57) ABSTRACT

Lu-containing compositions comprising a particle coated by a Lu-containing film are disclosed. The process of depositing the Lu-containing film on the particle is also disclosed.

### CROSS REFERENCE TO RELATED APPLICATIONS

The present application claims priority to U.S. Application Ser. No. 62/542,078 filed Aug. 7, 2017, herein incorporated by reference in its entirety for all purposes.

### TECHNICAL FIELD

Lu-containing compositions comprising a particle coated by a Lu-containing film are disclosed. The process of depositing the Lu-containing film on the particle is also disclosed.

### BACKGROUND

¹⁷⁷Lu has been tested for cancer treatment. See, e.g., Das et al., Cancer Biother Radiopharm, 2011 Jun.; 26 (3), 395-400. As the half-life of ¹⁷⁷Lu is 6.73 days, these treatments are typically prepared at the hospital using a neutron therapy reactor to generate the radioisotope from more stable isotopes, such as ¹⁷⁶Lu, ¹⁷⁵Lu, or ¹⁷⁶ Yb.

Lu₂O₃ powder having a particle size of 3 microns is commercially available. See, e.g., US Research Nanomaterials, Inc.

U.S. Pat. No. 5,300,281 to McMillan et al discloses radioactive compositions containing a calcific matrix and methods for using the compositions for therapeutic radiation treatment including rheumatoid arthritis.

US Pat App Pub No 2001/0047185 to Satz discloses radioactivatable compositions, preferably metal alloy compositions containing a metal having shape memory characteristics, and at least one radioactivatable isotope comprising a lanthanide series element or mixtures of lanthanide series elements or other suitable isotopes.

US Pat App Pub No 2003/0026989 to George et al. discloses particles having an ultrathin, conformal coating made using atomic layer deposition methods.

U.S. Pat. No. 6,716,353 to Mirzadeh et al discloses a method of separating lutetium from a solution containing Lu and Yb, particularly reactor-produced ¹⁷⁷Lu and ¹⁷⁷Yb.

US Pat App Pub No 2009/0302434 to Pallem et al. discloses methods and compositions for depositing rare earth metal-containing layers.

U.S. Pat. No. 9,119,887 to Day et al. disclose low density radioactive magnesium-aluminum-silicate microparticles that contain either samarium-yttrium, samarium, or lutetium as medical isotopes for radiotherapy and/or radioimaging.

RU Pat App Pub Nos 2542733 and 2624636 disclose methods of producing isotopes for radiation medicine.

A need remains for cost effective sources of ¹⁷⁷Lu.

### SUMMARY

Lu-containing film forming compositions are disclosed. The Lu-containing film forming compositions comprise a precursor selected from the group consisting of:
(a) LuX₃, wherein X is F, Cl, I, or Br;
(b) Lu(Cp)₃;
(c) Lu[R—N—C(R″)═N—R′]₃, wherein R, R′ and R″ are H or the defined hydrocarbyl group;
(d) Lu[—O—C(R)—C═C(R′)—O—′]₃, wherein R and R′ are H or the defined hydrocarbyl group;
(e) Lu[N(SiR₃)₂]₃, wherein each R is independently H or a hydrocarbyl group; and
(f) Lu(RCp)ₘ(R¹—N—C(R²)═N—R³)ₙ, wherein R, R¹, R², and R³ is H or a hydrocarbyl group.

The disclosed Lu-containing film forming compositions may comprise one or more of the following aspects:
the precursor being LuX₃, wherein X is F, Cl, I, or Br;
the precursor being LuCl₃;
the precursor being ¹⁷⁶LuCl₃;
the precursor being Lu(Cp)₃;
the precursor being ¹⁷⁶Lu(Cp)₃;
the precursor being Lu(N^{iPr} Me-amd)₃;
the precursor being ¹⁷⁶Lu(N^{iPr} Me-amd)₃;
the precursor being Lu(N^{Et} Me-amd)₃;
the precursor being ¹⁷⁶Lu(N^{Et} Me-amd)₃;
the precursor being Lu[N(SiMe₃)₂]₃;
the precursor being ¹⁷⁶Lu[N(SiMe₃)₂]₃;
the precursor being LuCp₂(N^{iPr} Me-amd);
the precursor being ¹⁷⁶LuCp₂(N^{iPr} Me-amd);
the precursor being LuCp₂(N^{Et} Me-amd);
the precursor being ¹⁷⁶LuCp₂(N^{Et} Me-amd);
the precursor being Lu(MeCp)₂(N^{iPr} Me-amd);
the precursor being ¹⁷⁶Lu(MeCp)₂(N^{iPr} Me-amd);
the precursor being Lu(MeCp)₂(N^{Et} Me-amd);
the precursor being ¹⁷⁶Lu(MeCp)₂(N^{Et} Me-amd);
the precursor being Lu(EtCp)₂(N^{iPr} Me-amd);
the precursor being ¹⁷⁶Lu(EtCp)₂(N^{iPr} Me-amd);
the precursor being Lu(EtCp)₂(N^{Et} Me-amd);
the precursor being ¹⁷⁶Lu(EtCp)₂(N^{Et} Me-amd);
the Lu-containing film forming composition comprising between approximately 95% w/w and approximately 100% w/w of the precursor;
the Lu-containing film forming composition comprising between approximately 5% w/w and approximately 50% w/w of the precursor;
the Lu-containing film forming composition comprising between approximately 0 ppmw and approximately 500 ppmw Al;
the Lu-containing film forming composition comprising between approximately 0 ppmw and approximately 500 ppmw As;
the Lu-containing film forming composition comprising between approximately 0 ppmw and approximately 500 ppmw Ba;
the Lu-containing film forming composition comprising between approximately 0 ppmw and approximately 500 ppmw Be;
the Lu-containing film forming composition comprising between approximately 0 ppmw and approximately 500 ppmw Bi;
the Lu-containing film forming composition comprising between approximately 0 ppmw and approximately 500 ppmw Cd;
the Lu-containing film forming composition comprising between approximately 0 ppmw and approximately 500 ppmw Ca;
the Lu-containing film forming composition comprising between approximately 0 ppmw and approximately 500 ppmw Cr;
the Lu-containing film forming composition comprising between approximately 0 ppmw and approximately 500 ppmw Co;
the Lu-containing film forming composition comprising between approximately 0 ppmw and approximately 500 ppmw Cu;
the Lu-containing film forming composition comprising between approximately 0 ppmw and approximately 500 ppmw Ga;
the Lu-containing film forming composition comprising between approximately 0 ppmw and approximately 500 ppmw Ge;
the Lu-containing film forming composition comprising between approximately 0 ppmw and approximately 500 ppmw Hf;
the Lu-containing film forming composition comprising between approximately 0 ppmw and approximately 500 ppmw Zr;
the Lu-containing film forming composition comprising between approximately 0 ppmw and approximately 500 ppmw In;
the Lu-containing film forming composition comprising between approximately 0 ppmw and approximately 500 ppmw Fe;
the Lu-containing film forming composition comprising between approximately 0 ppmw and approximately 500 ppmw Pb,
the Lu-containing film forming composition comprising between approximately 0 ppmw and approximately 500 ppmw Li;
the Lu-containing film forming composition comprising between approximately 0 ppmw and approximately 500 ppmw Mg;
the Lu-containing film forming composition comprising between approximately 0 ppmw and approximately 500 ppmw Mn;
the Lu-containing film forming composition comprising between approximately 0 ppmw and approximately 500 ppmw W;
the Lu-containing film forming composition comprising between approximately 0 ppmw and approximately 500 ppmw Ni;
the Lu-containing film forming composition comprising between approximately 0 ppmw and approximately 500 ppmw K;
the o Lu-containing film forming composition comprising between approximately 0 ppmw and approximately 500 ppmw Na;
the Lu-containing film forming composition comprising between approximately 0 ppmw and approximately 500 ppmw Sr;
the Lu-containing film forming composition comprising between approximately 0 ppmw and approximately 500 ppmw Th;
the Lu-containing film forming composition comprising between approximately 0 ppmw and approximately 500 ppmw Sn;
the Lu-containing film forming composition comprising between approximately 0 ppmw and approximately 500 ppmw Ti;
the Lu-containing film forming composition comprising between approximately 0 ppmw and approximately 500 ppmw U;
the Lu-containing film forming composition comprising between approximately 0 ppmw and approximately 500 ppmw V;
the Lu-containing film forming composition comprising between approximately 0 ppmw and approximately 500 ppmw Zn;
the Lu-containing film forming organosilane composition comprising between approximately 0 ppmw and approximately 500 ppmw Cl;
the Lu-containing film forming composition comprising between approximately 0 ppmw and approximately 500 ppmw Br;
the Lu-containing film forming composition comprising between approximately 0 ppmw and approximately 500 ppmw I;
the Lu-containing film forming composition comprising between approximately 0.0% w/w and 0.1% w/w tetrahydrofuran (THF);
the Lu-containing film forming composition comprising between approximately 0.0% w/w and 0.1% w/w ether;
the Lu-containing film forming composition comprising between approximately 0.0% w/w and 0.1% w/w pentane;
the Lu-containing film forming composition comprising between approximately 0.0% w/w and 0.1% w/w cyclohexane;
the Lu-containing film forming composition comprising between approximately 0.0% w/w and 0.1% w/w heptane; or
the Lu-containing film forming composition comprising between approximately 0.0% w/w and 0.1% w/w toluene.

Lu-containing compositions are also disclosed. The Lu-containing compositions comprise a particle coated by a Lu-containing film. The disclosed Lu-containing composition may comprise one or more of the following aspects:
the particle having neutron transparency;
the particle having a low mass attenuation coefficient μ/ρ for neutrons, wherein μ is the attenuation coefficient (1/m) and ρ is the density (kg/m³),
the particle having a mass attenuation coefficient μ/ρ ranging from approximately 10 m²/kg to approximately 1000 m²/kg;
the particle having a mass attenuation coefficient μ/ρ ranging from approximately 10 m²/kg to approximately 1000 m²/kg;
the particle being Al₂O₃;
the particle being ZrO₂;
the particle being zirconia-toughened alumina;
the particle being yttria-stabilized zirconia;
the particle having a particle size ranging from approximately 0.2 microns to approximately 100 microns;
the Lu-containing film being LuF₃;
the Lu-containing film being Lu(OH)₃;
the Lu-containing film being Lu₂O₃;
the Lu-containing film being ¹⁷⁶Lu₂O₃;
the Lu-containing film being LuN;
the Lu-containing film being LuON; or
the Lu-containing film having a thickness ranging from approximately 10 Å to approximately 10,000 Å.

Methods of depositing Lu-containing films on a substrate by vapor deposition methods are also disclosed. The vapor of any of the Lu-containing film forming compositions disclosed above is introduced into a reactor containing a substrate. At least part of the precursor is deposited onto the substrate to form the Lu-containing film on the substrate using an atomic layer deposition process. The disclosed method may include one or more of the following aspects:
the vapor deposition method being a thermal atomic layer deposition process;
the vapor deposition method being a plasma enhanced atomic layer deposition process;
The vapor deposition method comprising fluidizing the substrate in a fluid bed reactor and reacting the Lu-containing film forming composition disclosed above with the fluidized substrate;
introducing a reactant into the reactor;
the reactant being selected from the group consisting of O₂, O₃, H₂O, H₂O₂, NO, NO₂, a carboxylic acid, an alcohol, a diol, radicals thereof, and combinations thereof;
the reactant being F₂, HF, NF₃, radicals thereof, and combinations thereof;
the reactant being plasma treated oxygen;
the Lu-containing film being a lutetium oxide film;
the Lu-containing film being a lutetium fluoride film;
the Lu-containing film being a lutetium hydroxide film;
the Lu-containing film being a lutetium nitride film;
the Lu-containing film being a lutetium oxynitride film;
the substrate being a ZrO₂ particle;
the substrate being an Al₂O₃ particle;
the substrate being a zirconia-toughened alumina particle;
the substrate being an yttria-stabilized zirconia particle;
the particle having a particle size ranging from approximately 10 nm microns to approximately 100 microns;
the method forming a Lu-containing film having a thickness ranging from approximately 1 Å to approximately 10,000 Å,
the method forming a Lu-containing film having a thickness ranging from approximately 1 nm to 300 nm; or
the method forming a Lu-containing film having a thickness ranging from approximately 10 nm to approximately 100 nm.

### Notation and Nomenclature

Certain abbreviations, symbols, and terms are used throughout the following description and claims, and include:

As used herein, the indefinite article “a” or “an” means one or more.

As used herein, the terms “approximately” or “about” mean ±10% of the value stated.

As used herein, the term “particle” means a small object having a diameter that ranges from approximately 1 nanometer to approximately 500 microns, preferably from approximately 50 microns to approximately 300 microns, that behaves as a whole unit with respect to its transport and properties.

As used herein, the term “independently” when used in the context of describing R groups should be understood to denote that the subject R group is not only independently selected relative to other R groups bearing the same or different subscripts or superscripts, but is also independently selected relative to any additional species of that same R group. For example in the formula MR¹x (NR²R³)₍₄₋ₓ₎, where x is 2 or 3, the two or three R¹ groups may, but need not be identical to each other or to R² or to R³. Further, it should be understood that unless specifically stated otherwise, values of R groups are independent of each other when used in different formulas.

As used herein, the term “hydrocarbyl group” refers to a functional group containing carbon and hydrogen; the term “alkyl group” refers to saturated functional groups containing exclusively carbon and hydrogen atoms. The hydrocarbyl group may be saturated or unsaturated. Either term refers to linear, branched, or cyclic groups. Examples of linear alkyl groups include without limitation, methyl groups, ethyl groups, propyl groups, butyl groups, etc. Examples of branched alkyls groups include without limitation, t-butyl. Examples of cyclic alkyl groups include without limitation, cyclopropyl groups, cyclopentyl groups, cyclohexyl groups, etc.

As used herein, the abbreviation “Me” refers to a methyl group; the abbreviation “Et” refers to an ethyl group; the abbreviation “Pr” refers to a propyl group; the abbreviation “nPr” refers to a “normal” or linear propyl group; the abbreviation “iPr” refers to an isopropyl group; the abbreviation “Bu” refers to a butyl group; the abbreviation “nBu” refers to a “normal” or linear butyl group; the abbreviation “tBu” refers to a tert-butyl group, also known as 1,1-dimethylethyl; the abbreviation “sBu” refers to a sec-butyl group, also known as 1-methylpropyl; the abbreviation “iBu” refers to an iso-butyl group, also known as 2-methylpropyl; the term “amyl” refers to an amyl or pentyl group (i.e., a C5 alkyl group); the term “tAmyl” refers to a tert-amyl group, also known as 1,1-dimethylpropyl.

As used herein, the abbreviation “Cp” refers to cyclopentadienyl group; the abbreviation “Cp*” refers to a pentamethylcyclopentadienyl group; the abbreviation “TMS” refers to trimethylsilyl (Me₃Si—); and the abbreviation “TMSA” refers to bis(trimethylsilyl)amine [—N(SiMe₃)₂].

As used herein, the abbreviation “N^{R, R′} R″-amd” or N^{R}R″-amd when R═R′ refers to the amidinate ligand [R—N—C(R″)═N—R′], wherein R, R′ and R″ are defined alkyl groups, such as Me, Et, nPr, iPr, nBu, iBi, sBu or tBu; the abbreviation “N^{R, R′}-fmd” or N^{R}-fmd when R═R′ refers to the formidinate ligand [R—N—C(H)═N—R′], wherein R and R′ are defined alkyl groups, such as Me, Et, nPr, iPr, nBu, iBi, sBu or tBu; the abbreviation “N^{R, R′}, N^{R′,R′″}-gnd” or N^{R}, N^{R″}-gnd when R═R′ and R″═R′″ refers to the guanidinate ligand [R—N—C(NR″R′″)═NR′], wherein R, R′, R″ and R′″ are defined alkyl group such as Me, Et, nPr, iPr, nBu, iBi, sBu or tBu. Although depicted here as having a double bond between the C and N of the ligand backbone, one of ordinary skill in the art will recognize that the amidinate, formidinate and guanidinate ligands do not contain a fixed double bond. Instead, one electron is delocalized amongst the N—C—N chain.

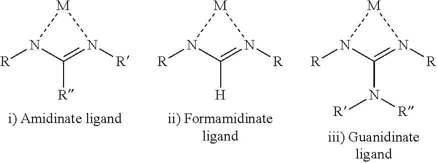

The standard abbreviations of the elements from the periodic table of elements are used herein. It should be understood that elements may be referred to by these abbreviations (e.g., Mn refers to manganese, Si refers to silicon, C refers to carbon, etc.). Additionally, Group 3 refers to Group 3 of the Periodic Table (i.e., Sc, Y, La, or Ac). Similarly, Group 4 refers to Group 4 of the Periodic Table (i.e., Ti, Zr, or Hf) and Group 5 refers to Group 5 of the Periodic Table (i.e., V, Nb, or Ta).

Any and all ranges recited herein are inclusive of their endpoints (i.e., x═1 to 4 or x ranges from 1 to 4 includes x=1, x=4, and x=any number in between), irrespective of whether the term “inclusively” is used.

Please note that the films or layers deposited, such as lutetium oxide or lutetium nitride, may be listed throughout the specification and claims without reference to their proper stoichiometry (i.e., Lu₂O₃). These films may also contain Hydrogen, typically from 0 at % to 15 at %. However, since not routinely measured, any film compositions given ignore their H content, unless explicitly stated otherwise.

A substrate is understood as the main solid material on which the film is deposited. It is understood that the film may be deposited on a stack of layers that are themselves on the substrate. Substrates are typically but not limited to wafers of silicon, glass, quartz, sapphire, GaN, AsGa, Ge. Substrates may be sheets, typically of metal, glass, organic materials like polycarbonate, PET, ABS, PP, HDPE, PMMA, etc. Substrates may be three-dimensional (3D) objects of similar materials, such as particles. On silicon wafers, typical layers over the substrate may be Ge, SiGe, silicon oxide, silicon nitride, metals (such as Cu, Co, Al, W, Ru, Ta, Ti, Ni), metal silicides and alloys, metal nitrides such as TaN, TiN, VN, NbN, HfN, VN; carbon doped silica films, whether dense or porous, silicon carbo-nitride, amorphous carbon, boron nitride, boron carbonitride, organic materials such as spin-on-carbon, polyim ides, photoresists and anti-reflective layers; metal oxides such as oxides of Ti, Hf, Zr, Ta, Nb, V, Mo, W, Al, and lanthanides. The substrates may have topographies like holes or trenches, typically having opening in the range of 5 nm to 100 μm, and usually between 20 nm and 1 μm, and aspect ratio of up to 1:1000, more usually in the range of 1:2 to 1:100.

### DESCRIPTION OF PREFERRED EMBODIMENTS

Lu-containing film forming compositions are disclosed. The Lu-containing film forming compositions comprise a precursor selected from:
(a) LuX₃, wherein X is F, Cl, I, or Br;
(b) Lu(Cp)₃;
(c) Lu[R—N—C(R″)═N—R′]₃, wherein R, R′ and R″ are H or the defined hydrocarbyl group;
(d) Lu[—O—C(R)—C═C(R′)—O—′]₃, wherein R and R′ are H or the defined hydrocarbyl group;
(e) Lu[N(SiR3)2]3, wherein each R is independently H or a hydrocarbyl group; or
(f) Lu(RCp)m(R¹—N—C(R²)═N—R³)n, wherein R, R¹, R², and R³ is H or a hydrocarbyl group.
These precursors are either commercially available or may be synthesized by methods known in the art.

LuCl₃ is commercially available. However, the solid form at standard temperature and pressure and potential for Cl⁻ impurities may limit the applicability of this precursor.

U.S. Pat. No. 4,882,206 discloses that Lu(Cp)₃ may be synthesized by reacting 3 equivalents of NaCp with one equivalent of LuCl₃ to produce LuCp₃ and 3 equivalents of NaCl. Preferably, ¹⁷⁶LuCl₃ is used as the starting material to produce ¹⁷⁶LuCp₃. ¹⁷⁶LuCl₃ is commercially available. Based on the teachings of U.S. Pat. No. 4,882,206, one of ordinary skill in the art will recognize that the cyclopentadienyl ligand may or may not be substituted by one or more hydrocarbyl groups. Exemplary precursors include, but are not limited to, Lu(Cp)₃, Lu(MeCp)₃, Lu(EtCp)₃, Lu(iPrCp)₃, Lu(iPr₃Cp)₃, Lu(MesCp)₃, or Lu(TMS—Cp)₃.

Lu[R—N—C(R″)═N—R′]₃ may be synthesized by reacting N,N′-dialkylcarbodiimide with LiMe₃. Preferably, ¹⁷⁶LuMe₃ is used as the starting material to produce ¹⁷⁶Lu[R—N—C(R″)═N—R′]₃. Exemplary precursors include, but are not limited to, Lu(N^{iPr} Me-amd)₃ or Lu(N^{Et} Me-amd)₃.

Lu[—O—C(R)—C═C(R′)—O—′]₃ may be synthesized by reacting tris(isopropoxy) yttrium with 2,4-pentadione. tris(isopropoxy) lmyttrium is used as the starting material to produce ¹⁷⁶Lu[—O—C(R)—C═C(R′)—O—′]₃. Exemplary precursors include, but are not limited to, Lu(acac)₃ or Lu(hfac)₃.

Lu[N(SiR₃)₂]₃ may be synthesized by reacting one equivalent of LuCl₃ with 3 equivalents of Na[N(SiMe₃)₂]. Preferably, ¹⁷⁶LuCl₃ is used as the starting material to produce ¹⁷⁶ Lu[N(SiR₃)₂]₃. ¹⁷⁶LuCl₃ is commercially available. Exemplary precursors include, but are not limited to, Lu(TMSA)3, Lu[N(SiMe₂H)₂]₃, or Lu[N(SiEt₃)₂]₃.

Lu(RCp)ₘ(R¹—N—C(R²)═N—R³)ₙ may be synthesized by reacting LuCp₃ with one equivalent of amidine/guanidine to yield LuCp₂(amd/gnd) or with two equivalents of amidine/guanidine to yield LuCp(amd/gnd)₂. Preferably, ¹⁷⁶LuCp₃ is used as the starting material to produce ¹⁷⁶ Lu(RCp)ₘ(R¹—N—C(R²)═N—R³)ₙ. Exemplary precursors include, but are not limited to, LuCp₂(N^{iPr} Me-amd), LuCp₂(N^{Et} Me-amd), Lu(MeCp)₂(N^{iPr} Me-amd), Lu(MeCp)₂(N^{Et} Me-amd), Lu(EtCp)₂(N^{iPr} Me-amd), or Lu(EtCp)₂(N^{Et} Me-amd). These precursors are particularly preferred for the present applications due to their liquid form at standard temperature and pressure. The liquid precursor is easier to handle as compared to the solid precursors and is well suited for use in a fluid bed deposition process.

The ¹⁷⁶Lu isotope is more stable than ¹⁷⁷Lu and may be purchased commercially. Applicants believe that precursors synthesized using the ¹⁷⁶Lu isotope may provide more effective production of ¹⁷⁷Lu than those synthesized with the natural abundance Lu reactants.

To ensure process reliability, the disclosed Lu-containing film forming compositions may be purified by continuous or fractional batch distillation prior to use to a purity ranging from approximately 95% w/w to approximately 100% w/w, preferably ranging from approximately 98% w/w to approximately 100% w/w. One of ordinary skill in the art will recognize that the purity may be determined by H NMR or gas or liquid chromatography with mass spectrometry. The Lu-containing film forming composition may contain any of the following impurities: halides (X₂), trisilylamine, monohalotrisilylamine, dihalotrisilylamine, SiH₄, SiH₃X, SnX₂, SnX₄, HX, NH₃, NH₃X, monochlorosilane, dichlorosilane, alcohol, alkylamines, dialkylamines, alkylimines, THF, ether, pentane, cyclohexane, heptanes, or toluene, wherein X is Cl, Br, or I. Preferably, the total quantity of these impurities is below 0.1% w/w. The purified composition may be produced by recrystallisation, sublimation, distillation, and/or passing the gas or liquid through a suitable adsorbent, such as a 4A molecular sieve or a carbon-based adsorbent (e.g., activated carbon). Optionally, the composition may be filtered to reach specifications that are typical of products used in the semiconductor industry.

Purification of the disclosed Lu-containing film forming composition may also produce concentrations of trace metals and metalloids ranging from approximately 0 ppmw to approximately 500 ppmw, and more preferably from approximately 0 ppmw to approximately 100 ppmw. These metal or metalloid impurities include, but are not limited to, Aluminum(Al), Arsenic(As), Barium(Ba), Beryllium(Be), Bismuth(Bi), Cadmium(Cd), Calcium(Ca), Chromium(Cr), Cobalt(Co), Copper(Cu), Gallium(Ga), Germanium(Ge), Hafnium(Hf), Zirconium(Zr), Indium(In), Iron(Fe), Lead(Pb), Lithium(Li), Magnesium(Mg), Manganese(Mn), Tungsten(W), Nickel(Ni), Potassium(K), Sodium(Na), Strontium(Sr), Thorium(Th), Tin(Sn), Titanium(Ti), Uranium(U), Vanadium(V) and Zinc(Zn). The concentration of X (where X═Cl, Br, I) in the purified Lu-containing film forming compositions may range between approximately 0 ppmw and approximately 100 ppmw and more preferably between approximately 0 ppmw to approximately 10 ppmw.

The concentration of each solvent (such as THF, ether, pentane, cyclohexane, heptanes, and/or toluene) in the purified Lu-containing film forming compositions may range from approximately 0% w/w to approximately 5% w/w, preferably from approximately 0% w/w to approximately 0.1% w/w. Solvents may be used in the precursor composition's synthesis. Separation of the solvents from the precursor composition may be difficult if both have similar boiling points. Cooling the mixture may produce solid precursor in liquid solvent, which may be separated by filtration. Vacuum distillation may also be used, provided the precursor composition is not heated above approximately its decomposition point.

Alternatively, the disclosed Lu-containing film forming compositions may further comprise a solvent, such as fluorinated solvents, ethyl benzene, xylene, mesitylene, decane, and/or dodecane. The solvent should not react with the precursor. The disclosed precursors may be present in varying concentrations in the solvent. When the precursors Lu-containing film forming compositions include a solvent, the stability and vapor delivery performance of the precursor may be improved.

The Lu-containing film forming compositions are used to form the disclosed Lu-containing compositions. The Lu-containing compositions comprise a particle coated by a Lu-containing film. The particle has neutron transparency and a low mass attenuation coefficient μ/ρ for neutrons, wherein μ is the attenuation coefficient (1/m) and p is the density (kg/m³). A low mass attenuation coefficient μ/ρ may be defined as a value where μ/ρ/d>>1, wherein d is the diameter of the particle. A value of μ/ρ/d of at least 10 is needed and preferably at least 100.

Exemplary particles are commercially available and include Al₂O₃, ZrO₂, zirconia-toughened alumina, or yttria-stabilized zirconia. The particles have a particle size ranging from approximately 0.05 microns to approximately 500 microns, preferably from approximately 0.2 microns to approximately 100 microns. The Lu-containing film has a thickness ranging from approximately 10 Å to approximately 10,000 Å. The Lu-containing film is LuF₃, ¹⁷⁶LuF₃, ¹⁷⁷LuF₃, Lu(OH)₃, ¹⁷⁶Lu(OH)₃, ¹⁷⁷Lu(OH)₃, Lu₂O₃, ¹⁷⁶Lu₂O₃, and combinations thereof. The Lu-containing film has the same isotopic ratio of Lu contained in the precursor. The Lu precursor my initially predominantly contain enriched ¹⁷⁶Lu. For example, the precursor may initially contain between approximately 90% w/w to approximately 100% w/w of the ¹⁷⁶Lu compound. After irradiation with neutrons and for a short period of time (i.e., 6.7 days or less, which is the half life of ¹⁷⁷Lu), the Lu-containing film may predominantly contain a combination of the ¹⁷⁶Lu and ¹⁷⁷Lu isotopes. For example, the Lu-containing film may contain between approximately 90% w/w to approximately 100% w/w of the ¹⁷⁷Lu compound.

The disclosed Lu-containing film forming compositions may provide improved yield of ¹⁷⁷Lu. More particularly, less Lu-containing film forming composition is required to prepare the Lu-containing composition as compared to the amount of material necessary to produce powdered Lu₂O₃. As a result, coating the neutron transparent particle with the Lu-containing film provides a more efficient use of the ¹⁷⁶Lu-containing raw materials.

Applicants further believe that production of ¹⁷⁷Lu radioisotopes from the disclosed Lu-containing compositions will be more effective than its production from commercially available pure Lu₂O₃ powders. The effectiveness comes from the fact that the neutron irradiation has a limited penetration depth into the Lu₂O₃ and therefore the ¹⁷⁶Lu to ¹⁷⁷Lu nuclear reaction that is desired does not occur throughout the entire diameter of the Lu₂O₃ sphere. As a result, longer neutron beam exposures are required to have an adequate overall transformation of the ¹⁷⁶Lu to the desired ¹⁷⁷Lu.

RU Published App No 2594020 discloses the usage of ¹⁷⁶Lu₂O₃ particles having ˜20 nm diameters as a raw material to form ¹⁷⁷Lu₂O₃. The 20 nm particle size is selected so the lamda/d >>1 as described in the publication. The handling of 20 nm size particles is highly problematic because of extremely low settling velocities, ease of fluidization, and an overlap lack of an ability to manipulate the nanopowder material. Using larger diameter spheres made up of a neutron beam transparent core with a nanometer thickness coating of a ¹⁷⁶Lu containing film overcomes the handling difficulties and allows for the highly effective transformation of the ¹⁷⁶Lu to ¹⁷⁷Lu. Commercially available Lu₂O₃ powders lose effectiveness because the requirement for lamda/d>>1 is no longer satisfied and the transformation of the ¹⁷⁶Lu to ¹⁷⁷Lu is not effectively performed within the core of the particle.

The disclosed Lu-containing compositions are prepared using a vapor deposition process. More particularly, the vapor of any of the Lu-containing film forming compositions disclosed above is introduced into a reactor containing a substrate. At least part of the precursor is deposited onto the substrate to form the Lu-containing film on the particle using an atomic layer deposition process.

The Lu-containing film forming compositions are introduced into a reactor in vapor form by conventional means, such as tubing and/or flow meters. The vapor form may be produced by vaporizing the composition through a conventional vaporization step such as direct vaporization, distillation, or by bubbling, or by using a sublimator such as the one disclosed in PCT Publication WO2009/087609 to Xu et al. The composition may be fed in a liquid state to a vaporizer where it is vaporized before it is introduced into the reactor. Alternatively, the composition may be vaporized by passing a carrier gas into a container containing the compound or by bubbling the carrier gas into the compound. The carrier gas may include, but is not limited to, Ar, He, N₂, and mixtures thereof. Bubbling with a carrier gas may also remove any dissolved oxygen present in the neat or blended compound solution. The carrier gas and vapor form of the composition are then introduced into the reactor as a vapor.

If necessary, the container may be heated to a temperature that permits the composition to be in its liquid phase and to have a sufficient vapor pressure. The container may be maintained at temperatures in the range of, for example, approximately 50° C. to approximately 180° C. Those skilled in the art recognize that the temperature of the container may be adjusted in a known manner to control the amount of composition vaporized.

The reaction chamber may be any enclosure or chamber of a device in which deposition methods take place, such as, without limitation, a parallel-plate type reactor, a cold-wall type reactor, a hot-wall type reactor, a single-wafer reactor, a multi-wafer reactor, or other such types of deposition systems. All of these exemplary reaction chambers are capable of serving as an ALD reaction chamber. The reaction chamber may be maintained at a pressure ranging from about 0.5 mTorr to about 20 Torr, preferably between about 0.1 Torr and about 5 Torr. In addition, the temperature within the reaction chamber may range from about 50° C. to about 600° C. One of ordinary skill in the art will recognize that the optimal deposition temperature range for each Lu-containing film forming compositions may be determined experimentally to achieve the desired result.

In one preferred embodiment, the reactor may be a fluidized bed reactor. Methods of fluidizing particulate matter are well known and include passing a fluidizing gas, such as N₂, Ar, He, Ne, Xe, Kr, and mixtures thereof, upward through a porous plate or screen containing the material to be fluidized. The Lu-containing film forming compositions and any reactants may be introduced into the fluidized stream of particles. Any reactants may additionally be treated by plasma to generate radicals.

The reactor contains one or more substrates onto which the thin films will be deposited. A substrate is generally defined as the material on which a process is conducted. As discussed above, the substrate may be a particle. Alternatively, the substrates may be any suitable substrate used in semiconductor, photovoltaic, flat panel, or LCD-TFT device manufacturing. Examples of suitable substrates include wafers, such as silicon, SiGe, silica, glass, or Ge. Plastic substrates, such as poly(3,4-ethylenedioxythiophene)poly (styrenesulfonte) [PEDOT:PSS], may also be used. The substrate may also have one or more layers of differing materials already deposited upon it from a previous manufacturing step. For example, the wafers may include silicon layers (crystalline, amorphous, porous, etc.), silicon oxide layers, silicon nitride layers, silicon oxy nitride layers, carbon doped silicon oxide (SiCOH) layers, or combinations thereof. Additionally, the wafers may include copper, cobalt, ruthenium, tungsten and/or other metal layers (e.g. platinum, palladium, nickel, ruthenium, or gold). The wafers may include barrier layers or electrodes, such as tantalum, tantalum nitride, etc. Plastic layers, such as poly(3,4-ethylenedioxythiophene)poly (styrenesulfonate) [PEDOT:PSS] may also be used. The layers may be planar or patterned. The substrate may be an organic patterned photoresist film. The substrate may include layers of oxides which are used as dielectric materials in MIM, DRAM, or FeRam technologies (for example, ZrO₂ based materials, HfO₂ based materials, TiO₂ based materials, rare earth oxide based materials, ternary oxide based materials, etc.) or from nitride-based films (for example, TaN, TiN, NbN) that are used as electrodes. The disclosed processes may deposit the Group IV-containing layer directly on the wafer or directly on one or more than one (when patterned layers form the substrate) of the layers on top of the wafer. Furthermore, one of ordinary skill in the art will recognize that the terms “film” or “layer” used herein refer to a thickness of some material laid on or spread over a surface and that the surface may be a trench or a line. Throughout the specification and claims, the wafer and any associated layers thereon are referred to as substrates. The actual substrate utilized may also depend upon the specific precursor embodiment utilized. In many instances though, the preferred substrate utilized will be selected from TiN, NbN, Ru, Si, and SiGe type substrates, such as polysilicon or crystalline silicon substrates. For example, a Group 4 metal oxide film may be deposited onto a TiN substrate. In subsequent processing, a TiN layer may be deposited on the Group 4 metal oxide layer, forming a TiN/Group 4 metal oxide/TiN stack used as DRAM capacitor. The Metal Oxide layer itself may be made of a stack of several layers of various metal oxides, generally selected from Group 4 metal oxide, Group 5 metal oxide, Al₂O₃, SiO₂, and MoO₂.

The temperature and the pressure within the reactor are held at conditions suitable for vapor depositions. In other words, after introduction of the vaporized composition into the chamber, conditions within the chamber are such that at least part of the vaporized precursor is deposited onto the substrate to form the Lu-containing film. For instance, the pressure in the reactor may be held between about 1 Pa and about 10⁶ Pa, more preferably between about 25 Pa and about 5×10³ Pa, as required per the deposition parameters. Likewise, the temperature in the reactor may be held between about 50° C. and about 500° C., preferably between about 100° C. and about 450° C. One of ordinary skill in the art will recognize that “at least part of the vaporized precursor is deposited” means that some or all of the precursor reacts with or adheres to the substrate.

The temperature of the reactor may be controlled by either controlling the temperature of the substrate holder or controlling the temperature of the reactor wall. Devices used to heat the substrate are known in the art. The reactor wall is heated to a sufficient temperature to obtain the desired film at a sufficient growth rate and with desired physical state and composition. A non-limiting exemplary temperature range to which the reactor wall may be heated includes from approximately 50° C. to approximately 500° C. When a plasma deposition process is utilized, the deposition temperature may range from approximately 50° C. to approximately 400° C. Alternatively, when a thermal process is performed, the deposition temperature may range from approximately 200° C. to approximately 450° C.

In addition to the disclosed Lu-containing film forming compositions, a reactant may also be introduced into the reactor. The reactant may be an oxidizing gas such as one of O₂, O₃, H₂O, H₂O₂, NO, N₂O, NO₂, a diol (such as ethylene glycol or hydrated hexafluoroacetone), an alcohol, such as ethanol or isopropanol, oxygen containing radicals such as O·or OH·, NO, NO₂, carboxylic acids, formic acid, acetic acid, propionic acid, and mixtures thereof. Preferably, the oxidizing gas is selected from the group consisting of O₂, O₃, H₂O, H₂O₂, oxygen containing radicals thereof such as O· or OH·, and mixtures thereof.

Alternatively, the reactant may be H₂, NH₃, hydrazines (such as N₂H₄, MeHNNH₂, Me₂NNH₂, MeHNNHMe, phenyl hydrazine), organic amines (such as NMeH₂, NEtH₂, NMe₂H, NEt₂H, NMe₃, NEt₃, (SiMe₃)₂NH, cyclic amines like pyrrolidine or pyrimidine), diamines (such as ethylene diamine, dimethylethylene diamine, tetramethylethylene diamine), aminoalcohols (such as ethanolamine [HO—CH₂—CH₂—NH₂], bis ethanolamine [HN(C₂H₅OH)₂] or tris ethanolamine[N(C₂H₅OH)₃]), pyrazoline, pyridine, radicals thereof, or mixtures thereof. Preferably the reactant is H₂, NH₃, radicals thereof, or mixtures thereof.

In another alternative, the reactant may be F₂, HF, NF₃, radicals thereof, and combinations thereof.

The reactant may be treated by a plasma, in order to decompose the reactant into its radical form. N₂ may also be utilized as a reducing gas when treated with plasma. For instance, the plasma may be generated with a power ranging from about 50 W to about 2500 W, preferably from about 100 W to about 400 W. The plasma may be generated or present within the reactor itself. Alternatively, the plasma may generally be at a location removed from the reactor, for instance, in a remotely located plasma system. One of skill in the art will recognize methods and apparatus suitable for such plasma treatment.

For example, the reactant may be introduced into a direct plasma reactor, which generates plasma in the reaction chamber, to produce the plasma-treated reactant in the reaction chamber. Exemplary direct plasma reactors include the Titan™ PECVD System produced by Trion Technologies. The reactant may be introduced and held in the reaction chamber prior to plasma processing. Alternatively, the plasma processing may occur simultaneously with the introduction of the reactant. In-situ plasma is typically a 13.56 MHz RF inductively coupled plasma that is generated between the showerhead and the substrate holder. The substrate or the showerhead may be the powered electrode depending on whether positive ion impact occurs. Typical applied powers in in-situ plasma generators are from approximately 30 W to approximately 1000 W. Preferably, powers from approximately 30 W to approximately 600 W are used in the disclosed methods. More preferably, the powers range from approximately 100 W to approximately 500 W. The disassociation of the reactant using in-situ plasma is typically less than achieved using a remote plasma source for the same power input and is therefore not as efficient in reactant disassociation as a remote plasma system, which may be beneficial for the deposition of Lu-containing films on substrates easily damaged by plasma.

Alternatively, the plasma-treated reactant may be produced outside of the reaction chamber. The MKS Instruments' ASTRONi® reactive gas generator may be used to treat the reactant prior to passage into the reaction chamber. Operated at 2.45 GHz, 7 kW plasma power, and a pressure ranging from approximately 0.5 Torr to approximately 10 Torr, the reactant O₂ may be decomposed into two O· radicals. Preferably, the remote plasma may be generated with a power ranging from about 1 kW to about 10 kW, more preferably from about 2.5 kW to about 7.5 kW.

The vapor deposition conditions within the chamber allow the disclosed Lu-containing film forming composition and the reactant to react and form a Lu-containing film on the substrate. In some embodiments, Applicants believe that plasma-treating the reactant may provide the reactant with the energy needed to react with the disclosed composition.

The Lu-containing film forming compositions and reactants may be introduced into the reactor either simultaneously (chemical vapor deposition), sequentially (atomic layer deposition) or different combinations thereof. The reactor may be purged with an inert gas between the introduction of the composition and the introduction of the reactant. Alternatively, the reactant and the composition may be mixed together to form a reactant/compound mixture, and then introduced to the reactor in mixture form. Another example is to introduce the reactant continuously and to introduce the Lu-containing film forming composition by pulse (pulsed chemical vapor deposition).

The vaporized composition and the reactant may be pulsed sequentially or simultaneously (e.g. pulsed CVD) into the reactor. Each pulse of composition may last for a time period ranging from about 0.01 seconds to about 100 seconds, alternatively from about 0.3 seconds to about 30 seconds, alternatively from about 0.5 seconds to about 10 seconds. The reactant may also be pulsed into the reactor. In such embodiments, the pulse of each gas may last from about 0.01 seconds to about 100 seconds, alternatively from about 0.3 seconds to about 30 seconds, alternatively from about 0.5 seconds to about 10 seconds. In another alternative, the vaporized composition and one or more reactants may be simultaneously sprayed from a shower head under which a susceptor holding several wafers is spun (spatial ALD).

Depending on the particular process parameters, deposition may take place for a varying length of time. Generally, deposition may be allowed to continue as long as desired or necessary to produce a film with the necessary properties. Typical film thicknesses may vary from several angstroms to several hundreds of microns, depending on the specific deposition process. The deposition process may also be performed as many times as necessary to obtain the desired film.

In one non-limiting exemplary CVD type process, the vapor phase of the disclosed Lu-containing film forming composition and a reactant are simultaneously introduced into the reactor. The two react to form the resulting Lu-containing thin film. When the reactant in this exemplary CVD process is treated with a plasma, the exemplary CVD process becomes an exemplary PECVD process. The reactant may be treated with plasma prior or subsequent to introduction into the chamber.

In one non-limiting exemplary ALD type process, the vapor phase of the disclosed Lu-containing film forming composition is introduced into the reactor, where the precursor physi- or chemisorbs on the substrate. Excess composition may then be removed from the reactor by purging and/or evacuating the reactor. A desired gas (for example, O₃) is introduced into the reactor where it reacts with the physi- or chemisorped precursor in a self-limiting manner. Any excess reducing gas is removed from the reactor by purging and/or evacuating the reactor. If the desired film is a Lu metal film, this two-step process may provide the desired film thickness or may be repeated until a film having the necessary thickness has been obtained.

Alternatively, if the desired film contains the Lu metal and a second element, the two-step process above may be followed by introduction of the vapor of an additional precursor compound into the reactor. The additional precursor compound will be selected based on the nature of the Lu film being deposited. After introduction into the reactor, the additional precursor compound is contacted with the substrate. Any excess precursor compound is removed from the reactor by purging and/or evacuating the reactor. Once again, a desired gas may be introduced into the reactor to react with the precursor compound. Excess gas is removed from the reactor by purging and/or evacuating the reactor. If a desired film thickness has been achieved, the process may be terminated. However, if a thicker film is desired, the entire four-step process may be repeated. By alternating the provision of the Lu-containing film forming composition, additional precursor compound, and reactant, a film of desired composition and thickness can be deposited.

When the reactant in this exemplary ALD process is treated with a plasma, the exemplary ALD process becomes an exemplary PEALD process. The reactant may be treated with plasma prior or subsequent to introduction into the chamber.

The Lu-containing films resulting from the processes discussed above may include a Lu oxide (LuᵢOₓ, wherein i ranges from 1 to 4 and x ranges from 1 to 6), a Lu fluoride (LuᵢFₓ, wherein i ranges from 1 to 4 and x ranges from 1 to 6), a Lu nitride(LuᵢNₓ, wherein i ranges from 1 to 4 and x ranges from 1 to 6), a Lu hydroxide (LuᵢOHₓ, wherein i ranges from 1 to 4 and x ranges from 1 to 6), or a Lu oxynitride (LuᵢN_{y}Oₓ, wherein i ranges from 1 to 4 and x and y range from 1 to 6). One of ordinary skill in the art will recognize that by judicial selection of the appropriate disclosed compositions, optional precursor compounds, and reactant species, the desired film composition may be obtained.

The resulting Lu-containing compositions may be used as a raw material for isotope transformation via neutron irradiation to form the ¹⁷⁷Lu-containing composition. The resulting Lu-containing composition is irradiated with neutrons to form a ¹⁷⁷Lu-containing composition. The ¹⁷⁷Lu isotope may be isolated from the Lu-containing composition using methods known in the art, including but not limited to solid phase extraction (see Ketrin, AR Production and Supply of High Specific Activity Radioisotopes for Radiotherapy Applications Alasbimn Journal 5 (19) January 2003), chromatography per U.S. Pat. No. 6,716,353, or dissolving the composition in a solvent and centrifuging per RU Pat App Pub No 2594020.

While embodiments of this invention have been shown and described, modifications thereof can be made by one skilled in the art without departing from the spirit or teaching of this invention. The embodiments described herein are exemplary only and not limiting. Many variations and modifications of the composition and method are possible and within the scope of the invention. Accordingly the scope of protection is not limited to the embodiments described herein, but is only limited by the claims which follow, the scope of which shall include all equivalents of the subject matter of the claims.

1. A Lu-containing composition comprising a particle coated by a radioisotopic Lu-containing film, the particle having neutron transparency.

2. The Lu-containing composition of claim 1, wherein the particle has a low mass attenuation coefficient μ/σ for neutrons, wherein μ is the attenuation coefficient (m²/kg) and a is the density (kg/m³).

3. The Lu-containing composition of claim 1, wherein the particle is selected from the group consisting of Al₂O₃, ZrO₂, zirconia-toughened alumina, yttria-stabilized zirconia, and combinations thereof.

4. The Lu-containing composition of claim 1, wherein the particle has a particle size ranging from approximately 0.2 microns to approximately 100 microns.

5. The Lu-containing composition of claim 1, wherein the radioisotopic Lu-containing film is selected from the group consisting of Lu₂O₃, LuF₃, Lu(OH)₃, and combinations thereof.

6. The Lu-containing composition of claim 1, wherein the radioisotopic Lu-containing film has a thickness ranging from approximately 10 Å to approximately 10,000 Å.

7. The Lu-containing composition of claim 1, wherein the radioisotopic Lu-containing film is formed via neutron irradiation.

8. A method of preparing the Lu-containing composition of claim 1, the method comprising introducing a vapor of a Lu-containing film forming compositions into a reactor containing the particle, wherein the Lu-containing film forming compositions comprises a precursor selected from the group consisting of:
(a) LuX₃, wherein X is F, Cl, I, or Br;
(b) Lu(Cp)₃;
(c) Lu[R—N—C(R″)═N—R′]₃, wherein R, R′ and R″ are H or the defined hydrocarbyl group;
(d) Lu[—O—C(R)—C═C(R′)—O—′]₃, wherein R and R′ are H or the defined hydrocarbyl group;
(e) Lu[N(SiR₃)₂]₃, wherein each R is independently H or a hydrocarbyl group; and
(f) Lu(RCp)ₘ(R¹—N—C(R²)═N—R³)ₙ, wherein R, R¹, R², and R³ is H or a hydrocarbyl group;
and depositing at least part of the precursor onto the particle to form the Lu-containing film on the particle using a vapor deposition process.

9. The method of claim 8, further comprising irradiating the Lu-containing composition with neutrons to form the radioisotopic Lu-containing film.

10. The method of claim 8, wherein the precursor comprises radioisotopic Lu.

11. The method of claim 8, wherein the vapor deposition method is selected from the group consisting of a thermal atomic layer deposition process, a plasma enhanced atomic layer deposition process, fluidizing the particles in a fluid bed reactor and reacting the Lu-containing film forming composition with the fluidized particles, and combinations thereof.

12. The method of claim 8, further comprising introducing a reactant into the reactor.

13. The method of claim 12, wherein the reactant is selected from the group consisting of O₂, O₃, H₂O, H₂O₂, NO, NO₂, a carboxylic acid, an alcohol, a diol, radicals thereof, and combinations thereof.

14. The method of claim 8, wherein the precursor is selected from the group consisting of LuCp₂(N^{iPr}Me-amd), LuCp₂(N^{Et}Me-amd), Lu(MeCp)₂(N^{iPr}Me-amd), Lu(MeCp)₂(N^{Et} Me-amd), Lu(EtCp)₂(N^{iPr} Me-amd), Lu(EtCp)₂(N^{Et} Me-amd), and combinations thereof.

15. The method of claim 14, wherein the precursor is Lu(EtCp)₂(N^{iPr} Me-amd).
